(19) United States
(12) Patent Application Publication
(10) Pub. No.: US 2007/0087439 A1
(43) Pub. Date: Apr. 19, 2007

(54) Method of measuring hexavalent chromium in electronic components and assemblies

(76) Inventors: Michael Riess, Taunusstein (DE); Heike Schumacher, Waldalgesheim (DE); Julia Smirnow, Wiesbaden (DE)

Correspondence Address: MOTOROLA, INC., 1303 EAST ALGONQUIN ROAD, IL01/3RD, SCHAUMBURG, IL, 60196, US

(21) Appl. No.: 11/249,634

(22) Filed: Oct. 13, 2005

(51) Int. Cl. G01N 33/20

(52) U.S. Cl. 436/83

(57) ABSTRACT

Hexavalent chromium in electronic components and assemblies is measured using x-ray fluorescence spectroscopy to analyze the sample to identify the matrix. Based on the ascertained matrix, a protocol is selected from a variety of extraction and analysis protocols, and the hexavalent chromium is extracted from the sample using the selected protocol. The extracted hexavalent chromium is reacted with 1,5 diphenylcarbazide and measured using ultraviolet spectroscopy using a unique calibration curve for each type of identified matrix. Based on the measured amount of hexavalent chromium, the concentration of hexavalent chromium is calculated as a function of a unit area of the sample.

### FIELD OF THE INVENTION

This invention relates generally to methods of measuring chromium. More particularly, this invention relates to methods of measuring the amount of hexavalent chromium in electronic components or electronic assemblies using x-ray fluorescence spectroscopy and ultraviolet spectroscopy.

### BACKGROUND

Legislation in the European Union (EU) has been enacted to reduce the level of hazardous chemicals in the environment. The Restriction of certain Hazardous Substances (RoHS) act has targeted materials such as chromium VI (also referred to as hexavalent chromium) used in electronic devices. In order to comply with these enacted regulations, a method to detect and identify this material in electronic devices is needed. Such a method should be fast, cost effective, and accurate to enable rapid testing and short turnaround times in keeping with the ‘time to market’ requirements of the global electronics industry. Although the methods of analysis for chromium VI in samples such as plating baths, wastewater, drinking water, and in the atmosphere have been well documented and yield highly accurate results, analysis of chromium VI in complex materials such as electronic assemblies and electronic components remains difficult and inaccurate. This is due to the highly complex and variable matrix of interfering materials in typical electronic products. Since prior art methods suffer from a lack of accuracy, an improved method for analyzing the amount of chromium VI in electronic components and assemblies would be a significant improvement to the art.

### BRIEF DESCRIPTION OF THE DRAWINGS

The features of the invention believed to be novel are set forth with particularity in the appended claims. The invention itself however, both as to organization and method of operation, together with objects and advantages thereof, may be best understood by reference to the following detailed description of the invention, which describes certain exemplary embodiments of the invention, taken in conjunction with the accompanying drawings in which:

FIG. 1 is a flow chart consistent with certain embodiments of the present invention.

FIGS. 2-7 are calibration curves consistent with certain embodiments of the present invention.

### DETAILED DESCRIPTION

While this invention is susceptible of embodiment in many different forms, there is shown in the drawings and will herein be described in detail specific embodiments, with the understanding that the present disclosure is to be considered as an example of the principles of the invention and not intended to limit the invention to the specific embodiments shown and described.

The disclosed embodiments employ X-ray fluorescence spectroscopy (XRF) and Ultraviolet (UV) spectroscopy as detecting methods, for measuring hexavalent chromium in electronic components and assemblies. The sample under test (an electronic component or assembly or equivalent) is analyzed using x-ray fluorescence spectroscopy in order to ascertain the matrix. A decision on how to treat the sample is then made, based on the identified matrix, i.e., the composition of the sample. If the matrix is aluminum, then the sample is extracted and analyzed to determine the amount of hexavalent chromium using a first parameter set. If the matrix is zinc, then the sample is extracted and analyzed to determine the amount of hexavalent chromium using a second parameter set. If the matrix is leather, then the sample is extracted and analyzed to determine the amount of hexavalent chromium using a third parameter set. If the matrix is plated steel, then the sample is extracted and analyzed to determine the amount of hexavalent chromium using a fourth parameter set. If the matrix is a printed wiring board or printed wiring assembly, then the sample is extracted and analyzed to determine the amount of hexavalent chromium using a fifth parameter set. Based on the analyzed amount of hexavalent chromium, the concentration of hexavalent chromium is calculated as a function of a unit area of the sample.

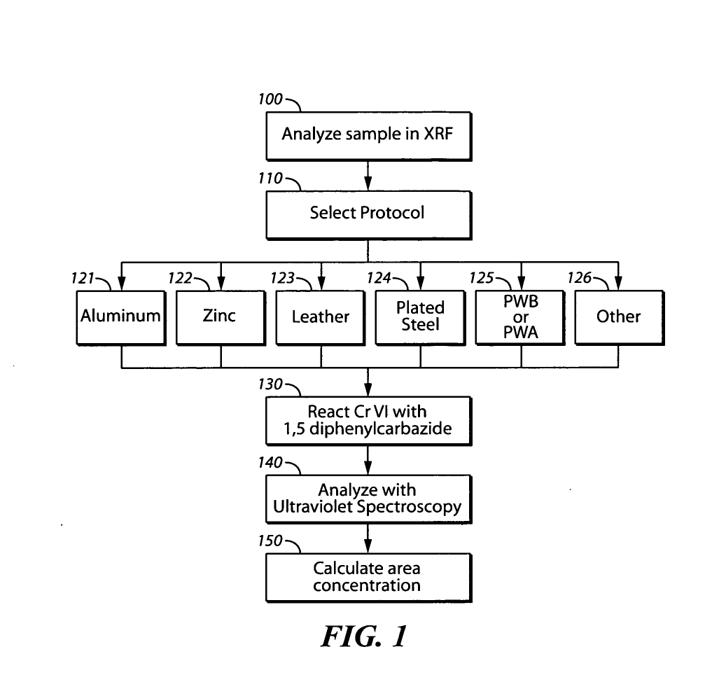

In the descriptions below, we utilize the terms “printed wiring assembly” (PWA) and “printed circuit board” (PCB) for convenience, but it is to be understood that use in this manner is not intended to be limiting, but is intended to cover these as well as other types of complex assemblies used in present and future electronic devices, such as, but not limited to, motors, cable assemblies, displays, switches, knobs, housings, speakers, transducers, etc. PCBs are well known in the industry, and conventionally are flat laminated structures that contain electrically conductive pathways that interconnect electronic components that are mounted thereon. PWAs conventionally refer to an assembly comprising the PCB and the components mounted thereon. Referring now to FIG. 1, a flow chart depicting one embodiment of the invention, the specimen or sample to be analyzed for presence of hexavalent chromium is placed directly in the chamber of a XRF analyzer (100). This method allows fast detection and identification because there is no sample preparation such as sputtering, that might use other environmentally critical materials. Examples of some materials commonly found in electronics that can be analyzed are leather (used for holsters and carrying cases), anti-corrosion coatings, plated steel, plated aluminum, zinc and zinc alloys, PCB, PWB, and other components. This listing is meant to be illustrative of some of the common materials that one may encounter, and is not intended to be limiting, as other materials commonly used in electronic assemblies can also be analyzed using our protocol. The XRF analysis indicates what type of materials are present in the sample, and based on this information, one then selects (110) an appropriate set of digestion and extraction parameters for treatment of the sample. The sample is removed from the XRF analyzer, and the sample is then digested according to the matrix type. The following are digestion protocols for several types of materials. In each case, all containers and solutions should be purged with an inert gas such as argon or nitrogen before and during use, and an inert gas blanket should be provided over the reaction vessels to prevent changing the oxidation state of the hexavalent chromium. Solvents should be treated with ultrasonic waves and purged with inert gas before use.

### Digestion of Plated Aluminum Samples

Using 25 ml of degassed water, add an amount of sample to equal approximately 50 cm² of plating, and boil on hot plate for 60 minutes once the solution has reached temperature. Cool to room temperature and add 500 microliter of phosphoric acid. Add water to bring to 25 ml volume.

### Digestion of Zinc Samples

Add 1 g NaOH, 0.1 g NaCO₃, 0.1 g MgCl₂·6H₂O to 50 ml of degassed water. Add an amount of sample to equal approximately 50 cm², and heat on hot plate for 10 minutes at 90-100° Centigrade once the solution has reached temperature. Test pH using pH paper. Filter through filter paper into a 25 ml flask. Add water to bring to volume.

### Digestion of Leather Samples

Use phosphate buffer at pH=8. Weigh 1.42 g NaH₂Po₄ (0.1 mole/liter) into a 250 ml Erlenmeyer flask and add 100 ml of degassed water. Purge with argon gas and add 1-5 drops of phosphoric acid. Test the pH using pH paper. Adjust accordingly until the pH is 8. Add 100 mg of the leather sample. Heat on hot plate for 10 minutes at 90-100° Centigrade once the solution has reached temperature. Reduce volume to 40 ml. Add phosphoric acid until pH<2. Test pH using pH paper. Filter through filter paper into a 50 ml flask. Add water to bring to volume.

### Digestion of Plated Steel Samples

Add 1 g KOH, 0.1 g NaCO₃, 0.1 g MgCl₂·6H₂O to 50 ml of degassed water. Add an amount of sample to equal approximately 50 cm², and heat on hot plate for 5 minutes at 90-100° Centigrade once the solution has reached temperature. Reduce volume to 20 ml, and add phosphoric acid drop wise until pH is 2±1. Precipitation may occur. Test pH using pH paper. Filter through filter paper into a 25 ml flask. Add water to bring to volume.

### Digestion of PWB or PWA Samples

Add 1 g KOH, 0.1 g NaCO₃, 0.1 g MgCl₂·6H₂O to 50 ml of degassed water. Add an amount of sample to equal approximately 100 mg of PWB material, and heat on hot plate for 5 minutes at 90-100° Centigrade once the solution has reached temperature. Reduce volume to 20 ml, and add phosphoric acid drop wise until pH is 2±1. Precipitation may occur. Test pH using pH paper. Filter through filter paper into a 25 ml flask. Add water to bring to volume.

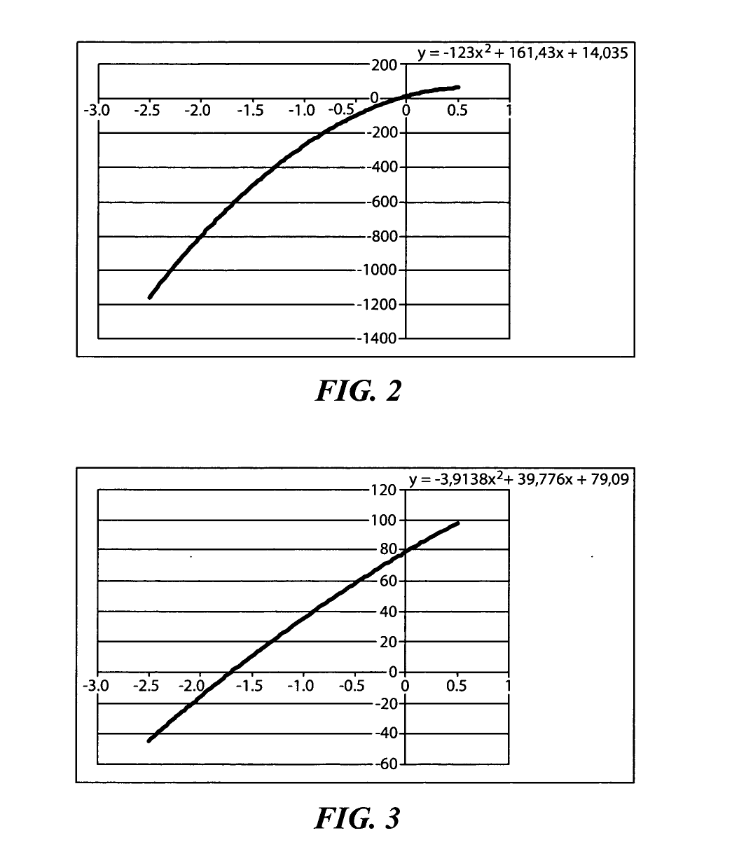

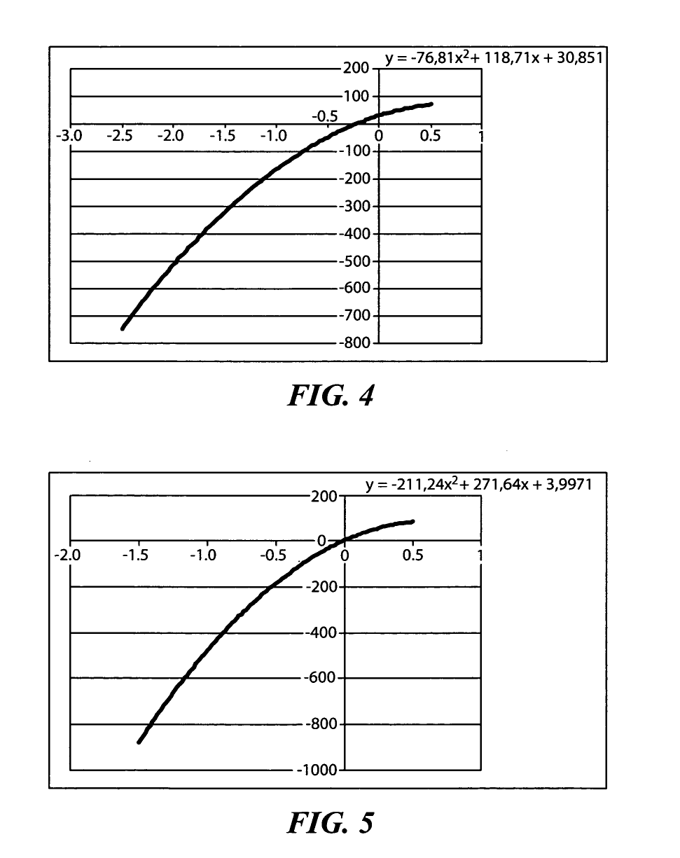

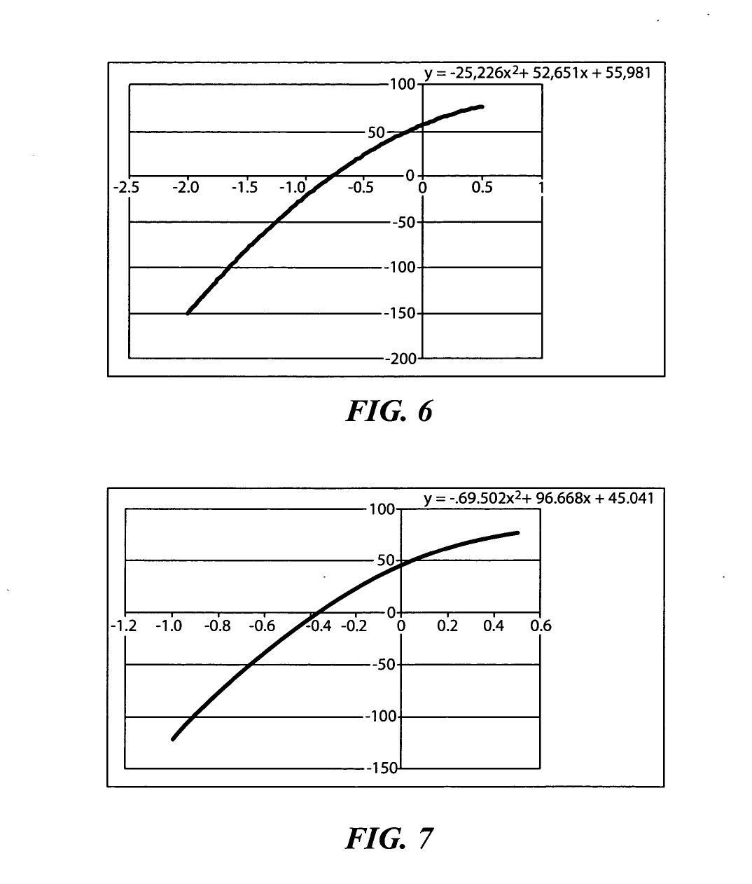

After the appropriate protocol has been selected (110), the sample is treated in accordance with the digestion method for one of the following: plated aluminum (121), zinc and zinc alloys (122), leather (123), plated steel (124), PCB or PWB (125), or other components (126). The hexavalent chromium in the digested and extracted sample is then reacted with 1,5 diphenylcarbazide (130) to convert it to 1,5 diphenylcarbazone and analyzed by ultraviolet spectroscopy (140). The amount of the red-violet complex is measured between 470 nanometers and 600 nanometers, with a analytical wavelength of 543 nanometers, for example, using conventional internal standard calibration methodology. Because the treatment methods for each type of matrix are different, a unique calibration curve is prepared for each matrix type in order to insure the highest levels of accuracy and reproducibility. In contrast to analysis methods for hexavalent chromium in, for example, wastewater, each matrix needs to have a unique calibration curve of intensity versus concentration, as shown in FIGS. 2-7. Prior art methods that utilize a single curve do not produce the accurate results that our novel method does. FIG. 2 is a calibration curve depicting the concentration of Cr VI (mg/l) as a function of background corrected absorption (%) at 543 nm for hexavalent Cr in an aluminum matrix, and FIGS. 3-6 are calibration curves for hexavalent Cr in a zinc matrix, a leather matrix, a plated steel matrix, and a PCB/PWA matrix, respectively. FIG. 7 is a calibration curve that was used with additional materials.

Once the concentration of hexavalent chromium per liter of solute is determined, then an additional step of calculating the amount of hexavalent chromium per unit area of the sample is performed (150). This step is critical because hexavalent chromium in electronic components and accessories is often found on the surface of the part or assembly, as in, for example, corrosion resistant coatings and cosmetic surface treatments. The area concentration of hexavalent chromium in the sample is then reported in micrograms per square centimeter. Other mass/unit area measuring schemes can also be used, in keeping with the numerical custom of the reporting country.

In summary, hexavalent chromium in electronic components and assemblies can be measured using x-ray fluorescence spectroscopy to analyze at least a portion of the sample in order to identify the matrix. Based on the ascertained matrix, a protocol is selected from a variety of extraction and analysis protocols, and the hexavalent chromium (if any) is extracted from the sample using the selected protocol. The extracted hexavalent chromium is reacted with 1,5 diphenylcarbazide and measured using ultraviolet spectroscopy using a unique calibration curve for each type of identified matrix. Based on the measured amount of hexavalent chromium, the concentration of hexavalent chromium is calculated as a function of a unit area of the sample.

While the invention has been described in conjunction with specific embodiments, it is evident that many alternatives, modifications, permutations and variations will become apparent to those of ordinary skill in the art in light of the foregoing description. For example, additional digestion and extraction protocols can be employed for other types of materials not mentioned here, such as plastics, glass displays, etc. Accordingly, it is intended that the present invention embrace all such alternatives, modifications and variations as fall within the scope of the appended claims.

What is claimed is:

1. A method of measuring hexavalent chromium in electronic components and assemblies, comprising:
providing a sample comprising an electronic component or assembly;
analyzing at least a portion of the sample using x-ray fluorescence spectroscopy in order to ascertain a matrix;
if the matrix is aluminum, then extracting and analyzing the amount of hexavalent chromium using a first parameter set;
if the matrix is zinc, then extracting and analyzing the amount of hexavalent chromium using a second parameter set;
if the matrix is leather, then extracting and analyzing the amount of hexavalent chromium using a third parameter set;
if the matrix is plated steel, then extracting and analyzing the amount of hexavalent chromium using a fourth parameter set;
if the matrix is a printed wiring board or printed wiring assembly, then extracting and analyzing the amount of hexavalent chromium using a fifth parameter set;
if the matrix is a material other than aluminum, zinc, leather, plated steel, printed wiring board or printed wiring assembly, then extracting and analyzing the amount of hexavalent chromium using a sixth parameter set; and
based on the analyzed amount of hexavalent chromium, calculating the concentration of hexavalent chromium as a function of a unit area of the sample.

2. The method as described in claim 1, wherein extracting using the first parameter set comprises extracting in water at a temperature greater than 90° C. and a pH of approximately 7.

3. The method as described in claim 1, wherein extracting using the second parameter set comprises extracting in water at a temperature greater than 90° C. and a pH of approximately 11.

4. The method as described in claim 1, wherein extracting using the third parameter set comprises extracting in phosphate buffered water at a temperature greater than 90° C. and a pH of approximately 8.

5. The method as described in claim 1, wherein extracting using the fourth parameter set comprises extracting in an aqueous KOH or NaOH solution at a temperature greater than 90° C.

6. The method as described in claim 1, wherein extracting using the fifth parameter set comprises extracting in water at a temperature greater than 90° C. and a pH of approximately 2.

7. The method as described in claim 1, wherein each of the first through sixth parameter sets comprises a unique calibration curve for analyzing.

8. The method as described in claim 7, wherein the hexavalent chromium is measured in each analysis protocol by reacting the extracted hexavalent chromium with 1,5 diphenylcarbazide and analyzing by ultraviolet spectroscopy.

9. The method as described in claim 1, wherein extracting is performed under an inert atmosphere.

10. A method of measuring hexavalent chromium in electronic components and assemblies, comprising:
providing a sample comprising an electronic component or assembly;
analyzing at least a portion of the sample using x-ray fluorescence spectroscopy in order to ascertain a matrix;
based on the ascertained matrix, selecting a protocol from the group consisting of aluminum extraction and analysis protocol, zinc extraction and analysis protocol, leather extraction and analysis protocol, steel extraction and analysis protocol, printed wiring board extraction and analysis protocol, and printed wiring assembly extraction and analysis protocol;
processing the sample using the selected protocol so as to extract and measure an amount of hexavalent chromium; and
based on the measured amount of hexavalent chromium, calculating the concentration of hexavalent chromium as a function of a unit area of the sample.

11. The method as described in claim 10, wherein the hexavalent chromium is measured in each analysis protocol by reacting the extracted hexavalent chromium with 1,5 diphenylcarbazide and analyzing by ultraviolet spectroscopy.

12. The method as described in claim 10, wherein the aluminum extraction and analysis protocol comprises extracting the sample in water at a temperature greater than 90° C. and a pH of approximately 7, and using an aluminum calibration curve to measure the amount of hexavalent chromium.

13. The method as described in claim 10, wherein the zinc extraction and analysis protocol comprises extracting the sample in water at a temperature greater than 90° C. and a pH of approximately 11, and using a zinc calibration curve to measure the amount of hexavalent chromium.

14. The method as described in claim 10, wherein the leather extraction and analysis protocol comprises extracting the sample in phosphate buffered water at a temperature greater than 90° C. and a pH of approximately 8, and using a leather calibration curve to measure the amount of hexavalent chromium.

15. The method as described in claim 10, wherein the steel extraction and analysis protocol comprises extracting the sample in an aqueous KOH or NaOH solution at a temperature greater than 90° C., and using a steel calibration curve to measure the amount of hexavalent chromium.

16. The method as described in claim 10, wherein the printed wiring board extraction and analysis protocol and the printed wiring assembly extraction and analysis protocol each comprises extracting the sample in water at a temperature greater than 90° C. and a pH of approximately 7, and using a printed wiring board calibration curve to measure the amount of hexavalent chromium.

17. The method as described in claim 10, wherein processing comprises extracting under an inert atmosphere.

18. A method of measuring hexavalent chromium in electronic components and assemblies, comprising:
providing a sample comprising an electronic component or assembly;
analyzing at least a portion of the sample using x-ray fluorescence spectroscopy in order to ascertain a matrix;
based on the ascertained matrix, selecting a protocol from the group consisting of aluminum extraction and analysis protocol, zinc extraction and analysis protocol, leather extraction and analysis protocol, steel extraction and analysis protocol, printed wiring board extraction and analysis protocol, and printed wiring assembly extraction and analysis protocol;
extracting hexavalent chromium from the sample using the selected protocol;
analyzing the extracted hexavalent chromium by complexing it with 1,5 diphenylcarbazide and measuring the complex using ultraviolet spectroscopy; and
based on the measured amount of hexavalent chromium, calculating the concentration of hexavalent chromium as a function of a unit area of the sample.

19. The method as described in claim 18, wherein extracting comprises extracting under an inert atmosphere.

20. The method as described in claim 18, wherein each selected extraction and analysis protocol comprises a unique calibration curve for measuring the complex.
